(19) United States
(12) Patent Application Publication
(10) Pub. No.: US 2004/0110710 A1
(43) Pub. Date: Jun. 10, 2004

(54) Methods of reducing ischemic injury

(76) Inventors: Xinkang Wang, Berwyn, PA (US); Gary Schieven, Lawrenceville, NJ (US); Giora Z. Feuerstein, Bryn Mawr, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/645,190

(22) Filed: Aug. 21, 2003

Related U.S. Application Data
(60) Provisional application No. 60/405,586, filed on Aug. 23, 2002.

(51) Int. Cl. A61K 48/00

(52) U.S. Cl. 514/44

(57) ABSTRACT

The present invention includes methods of reducing the activity, such as enzymatic activity and expression, of mitogen-activated protein kinase-activated protein kinase 2. The present invention further includes methods for identifying compounds useful for reducing such activity, and methods for reducing ischemic injury by the administration of such compounds.

This application claims the benefit of provisional U.S. application Ser. No. 60/405,586, filed Aug. 23, 2002, which is incorporated herein by reference in its entirety.

### FIELD OF THE INVENTION

The present invention includes methods of reducing the activity, such as enzymatic activity and expression, of mitogen-activated protein kinase-activated protein kinase 2. Particularly, the present invention includes methods for identifying compounds useful for reducing such activity, and methods for reducing ischemic injury by the administration of such compounds.

### BACKGROUND OF THE INVENTION

The three Mitogen-activated protein (MAP) kinase families include the extracellular regulated kinases (ERKs), the c-jun N-terminal kinase/stress-activated protein kinases (JNK/SAPKs) and the p38 MAP kinases. These MAP kinases have been implicated in a variety of cellular functions such as cell proliferation, differentiation and survival (Cano, E. and Mahadevan, L. C. Trends Biochem. Sci. 20:117-122 (1995)).

The p38 pathway is known to be important in the production of TNF-α, and an existing p38 inhibitor, SB203580, blocks TNF production and is active in animal models of acute and chronic inflammation as well as stroke and myocardial infarction. MAP kinase-activated protein kinase 2 (“MK2”) is one of several kinases that are regulated exclusively through direct phosphorylation by p38 MAP kinase in response to stress stimuli, and MK2 is an immediate down-stream kinase of the p38 MAP kinase signaling pathway. Mice deficient in MK2 show a reduction in bacterial lipopolysaccharide (LPS)-induced biosynthesis of tumor necrosis factor (TNF)-α, interferon (IFN)-γ, interleukin (IL)-1, IL-6 and nitric oxide (Kotlyarov et al., Nat. Cell Biol. 1:94-97 (1999)), suggesting a critical role of MK2 in inflammatory cytokine production.

Ischemia is a local anemia caused by an obstruction of blood supply to an organ or tissue. For example, myocardial ischemia results from an inadequate circulation of blood to the myocardium, usually as a result of coronary artery disease.

Cerebral ischemia is a pathophysiological condition caused by decrease in blood supply to the brain resulting in the deprivation of oxygen and glucose in the ischemic brain tissue, which eventually leads to cell death (necrosis and apoptosis) and inflammation (Wang, X. K. and Feuerstein, G. Z., Drug News Perspect. 13:133-140 (2000)). Concomitant activation of ERK, JNK and p38 MAP kinase has been reported in both gerbil and rat models of transient brain ischemia (Sugino et al., J. Neurosci 20:4506-4514 (2000); Irving et al., Mol. Brain Res. 77:65-75 (2000)), and certain MAP kinases have been implicated in cerebral ischemic injury.

For example, inhibition of ERK½ by a selective MEKI inhibitor revealed significant neuroprotection after transient cerebral ischemia in mice (Alessandrini et al., Proc. Natl. Acad. Sci. U.S.A. 96:12866-12869 (1999); Wang et al., Biochem. Biophys. Res. Commun. 286:869-874 (2001)). Inhibition of p38 MAP kinase reduced brain injury and neurological deficits following permanent occlusion of the middle cerebral artery (MCAO) in rats (Barone et al., J. Pharmacol. Exp. Ther. 296:312-321 (2001). However, inhibition of p38 results not only in a decrease in proinflammatory cytokines (TNF-α and IL-1β), but also, undesirably, in a decrease to anti-inflammatory cytokines (IL-10).

Accordingly, there exists a need to identify compounds useful for modulating the activity of targets associated with ischemia, which are useful for reducing the deleterious effects of ischemia. The present invention is directed to meeting such needs.

### SUMMARY OF THE INVENTION

The present invention includes methods of reducing the activity, such as enzymatic activity and expression, of mitogen-activated protein kinase-activated protein kinase 2. Particularly, the present invention includes methods for identifying compounds useful for reducing such activity, and methods for reducing ischemic injury by the administration of such compounds.

In one aspect, the present invention includes a method of reducing ischemic injury in a mammal, comprising administering to the mammal a compound that reduces activity, such as enzymatic activity and expression, of MK2. The ischemic injury may be, for example, cerebral ischemia, myocardial ischemia or critical limb ischemia.

In another aspect, the present invention includes a method of reducing ischemic injury in a mammal, comprising the steps of: (a) identifying a mammal suffering from ischemic injury; and (b) introducing to the mammal a compound that reduces expression of MK2. The compound may inhibit transcription of MK2, and may bind to a regulatory sequence operably linked to MK2. The compound may be an antisense nucleic acid, which may include at least 10 nucleotides, the sequence of which is complementary to an mRNA encoding an MK2 polypeptide. The antisense nucleic acid may be a DNA, wherein transcription of the DNA yields nucleic acid product which is complementary to an mRNA encoding an MK2 polypeptide.

In another aspect, the present invention includes a method of reducing ischemic injury in a mammal, comprising administering to the mammal an inhibitor of MK2 (e.g., mRNA and protein) expression.

In another aspect, the present invention includes a method of reducing ischemic injury in a mammal, comprising administering to the mammal a compound that reduces activity of MK2.

In another aspect, the present invention includes a method for identifying a compound which inhibits MK2 expression in a cell, the method comprising the steps of: (a) providing a cell that expresses MK2; (b) culturing the cell in the presence of a test compound; and (c) determining the level of expression of a MK2 in the cell, wherein a decrease in the level of expression in the presence of the test compound compared to the level of expression in the absence of the compound indicates that the test compound inhibits MK2 expression in the cell. The present invention also includes compounds, such as MK2 antagonists, identified by such a method.

In another aspect, the present invention includes an assay for identifying a compound that modulates the activity of MK2, including: (a) providing a cell expressing MK2; (b) contacting the cell expressing MK2 with a test compound; and (c) determining whether the test compound modulates the activity of MK2. The assay may be a cell-based assay or a cell-free assay. The cell-free assay may be a ligand-binding assay. The test compound may modulate the activity of MK2, and may be an MK2 antagonist or an MK2 agonist. Also, the test compound may bind to MK2. The assay may be for identifying compounds which will be useful for the treatment of ischemic injury. The present invention also includes compounds, such as MK2 antagonists, identified by such an assay.

In another aspect, the present invention includes a method for the treatment of ischemic injury, including administering to a patient in need thereof a therapeutically effective amount of a compound identified by an assay described above.

In another aspect, the present invention includes a method for the treatment of ischemic injury, including: (a) identifying a patient suffering from ischemic injury; and (b) administering to the patient a therapeutically effective amount of a modulator of MK2.

### BRIEF DESCRIPTION OF THE DRAWINGS

FIG. 1 shows infarct size in MK2^{-/-} and wild type mice following transient and permanent MCAO.

FIG. 2 shows the effect of transient MCAO on neurological deficits in MK2^{-/-} and wild type mice.

FIG. 3 shows the mRNA expression of IL-1β and TNFα in MK2^{-/-} and wild type mice after transient MCAO.

FIG. 4 shows the levels of IL-1β expression after transient and permanent MCAO in MK2^{-/-} and wild type mice.

FIG. 5A shows the expression of active caspase-3 (p20) in the brain of MK2^{-/-} and wild type mice after MCAO.

FIG. 5B shows DNA fragmentation in the brain of MK2^{-/-} and wild type mice after MCAO.

### DETAILED DESCRIPTION OF THE INVENTION

The present invention includes methods of reducing ischemia (e.g., cerebral ischemia) by modulating the activity, such as enzymatic activity and expression, of MK2. In the present invention, “reducing ischemia” means any amelioration of symptoms commonly associated with ischemia, including but not limited to decrease blood supply to a tissue or organ, deprivation of oxygen and glucose to said tissue or organ, cell death (necrosis and apoptosis), and inflammation. In the present invention, mice genetically deficient in MK2 were used to identify the effect of MK2 on ischemia, specifically cerebral ischemia, in both transient and permanent focal stroke induced by middle cerebral artery occlusion (“MCAO”). Histological and functional variables were explored along with biochemical markers of inflammation and apoptosis.

In the present invention, MK2 includes published MK2 sequences. By way of example only, MK2 useful in the present invention may be the transcript variant 1 having a nucleic acid sequence identified by Genbank Accession No. NM_{—}004759, set forth in Table 1, which encodes an isoform 1 protein having an amino acid sequence identified by Genbank Accession No. NP_{—}004750, set forth in Table 2.

TABLE 1
Nucleotide Sequence of MK2 Variant 1: Genbank Accession No. NM 004759

| | |
|---|---|
| 1 gatatcacag caacattgaa atgctaaaaa gtttttaaac actctcaatt tctaattcac | (SEQ ID NO:1) |
| 61 catgtcacag actggtgaaa aaaaaaaaaa aagcggccgc ttccccccgg ccgggccccc | |
| 121 gccgccccgc ggtccccaga gcgccaggcc cccgggggga gggagggagg gcgccgggcc | |
| 181 ggtgggagcc agcggcgcgc ggtgggaccc acggagcccc gcgacccgcc gagcctggag | |
| 241 ccgggccggc tcggggaagc cggctccagc ccggagcgaa cttcgcagcc cgtcgggggg | |
| 301 cggcggggag ggggcccgga gccggaggag ggggcggccg cgggcacccc cgcctgtgcc | |
| 361 ccggcgtccc cgggcaccat gctgtccaac tcccagggcc agagcccgcc ggtgccgttc | |
| 421 cccgccccgg ccccgccgcc gcagcccccc acccctgccc tgccgcaccc cccggcgcag | |
| 481 ccgccgccgc cgcccccgca gcagttcccg cagttccacg tcaagtccgg cctgcagatc | |
| 541 aagaagaacg ccatcatcga tgactacaag gtcaccagcc aggtcctggg gctgggcatc | |
| 601 aacggcaaag ttttgcagat cttcaacaag aggacccagg agaaattcgc cctcaaaatg | |
| 661 cttcaggact gccccaaggc ccgcagggag gtggagctgc actggcgggc ctcccagtgc | |
| 721 ccgcacatcg tacggatcgt ggatgtgtac gagaatctgt acgcagggag gaagtgcctg | |
| 781 ctgattgtca tggaatgttt ggacggtgga gaactcttta gccgaatcca ggatcgagga | |
| 841 gaccaggcat tcacagaaag agaagcatcc gaaatcatga agagcatcgg tgaggccatc | |
| 901 cagtatctgc attcaatcaa cattgcccat cgggatgtca agcctgagaa tctcttatac | |
| 961 acctccaaaa ggcccaacgc catcctgaaa ctcactgact ttggctttgc caaggaaacc | |
| 1021 accagccaca actctttgac cactccttgt tatacaccgt actatgtggc tccagaagtg | |
| 1081 ctgggtccag agaagtatga caagtcctgt gacatgtggt ccctgggtgt catcatgtac | |
| 1141 atcctgctgt gtgggtatcc ccccttctac tccaaccacg gccttgccat ctctccgggc | |
| 1201 atgaagactc gcatccgaat gggccagtat gaatttccca acccagaatg gtcagaagta | |
| 1261 tcagaggaag tgaagatgct cattcggaat ctgctgaaaa cagagcccac ccagagaatg | |
| 1321 accatcaccg agtttatgaa ccacccttgg atcatgcaat caacaaaggt ccctcaaacc | |
| 1381 ccactgcaca ccagccgggt cctgaaggag gacaaggagc ggtgggagga tgtcaagggg | |
| 1441 tgtcttcatg acaagaacag cgaccaggcc acttggctga ccaggttgtg agcagaggat | |
| 1501 tctgtgttcc tgtccaaact cagtgctgtt tcttagaatc cttttattcc ctgggtctct | |
| 1561 aatgggacct taaagaccat ctggtatcat cttctcattt tgcagaagag aaactgaggc | |
| 1621 ccagaggcgg agggcagtct gctcaaggtc acgcagctgg tgactggttg gggcagaccg | |
| 1681 gacccaggtt tcctgactcc tggcccaagt ctcttcctcc tatcctgcgg gatcactggg | |
| 1741 gggctctcag ggaacagcag cagtgccata gccaggctct ctgctgccca gcgctggggt | |
| 1801 gaggctgccg ttgtcagcgt ggaccactaa ccagcccgtc ttctctctct gctcccaccc | |
| 1861 ctgccgccct caccctgccc ttgttgtctc tgtctctcac gtctctcttc tgctgtctct | |
| 1921 cctacctgtc ttctggctct ctctgtaccc ttcctggtgc tgccgtgccc ceaggaggag | |
| 1981 atgaccagtg ccttggccac aatgcgcgtt gactacgagc agatcaagat aaaaaagatt | |
| 2041 gaagatgcat ccaaccctct gctgctgaag aggcggaaga aagctcgggc cctggaggct | |
| 2101 gcggctctgg cccactgagc caccgcgccc tcctgcccac gggaggacaa gcaataactc | |
| 2161 tctacaggaa tatatttttt aaacgaagag acagaactgt ccacatctgc ctcctctcct | |
| 2221 cctcagctgc atggagcctg gaactgcatc agtgactgaa ttc | |

TABLE 2
Amino Acid Sequence of MK2 Isoform 1: Genbank Accession No. NP 004750

| | |
|---|---|
| 1 mlsnsqgqsp pvpfpapapp pqpptpalph ppaqpppppp qqfpqfhvks glqikknaii | (SEQ ID NO:2) |
| 61 ddykvtsqvl glgingkvlq ifnkrtqekf alkmlqdcpk arrevelhwr asqcphivri | |
| 121 vdvyenlyag rkcllivmec ldggelfsri qdrgdqafte reaseimksi geaiqylhsi | |
| 181 niahrdvkpe nllytskrpn ailkltdfgf akettshnsl ttpcytpyyv apevlgpeky | |
| 241 dkscdmwslg vimyillcgy ppfysnhgla ispgmktrir mgqyefpnpe wsevseevkm | |
| 301 limllktep tqrmtitefm rihpwimqstk vpqtplhtsr vlkedkerwe dvkgclhdkn | |
| 361 sdqatwltrl | |

Also, by way of example only, MK2 useful in the present invention may be the transcript variant 2 having a nucleic acid sequence identified by Genbank Accession No. NM_{—}032960, set forth in Table 3, which encodes an isoform 2 protein having an amino acid sequence identified by Genbank Accession No. NP_{—}116584, set forth in Table 4.

TABLE 3
Nucleotide Sequence of MK2 Variant 2: Genbank Accession No. NM 032960

| | |
|---|---|
| 1 gatatcacag caacattgaa atgctaaaaa gtttttaaac actctcaatt tctaattcac | (SEQ ID NO:3) |
| 61 catgtcacag actggtgaaa aaaaaaaaaa aagcggccgc ttccccccgg ccgggccccc | |
| 121 gccgccccgc ggtccccaga gcgccaggcc cccgggggga gggagggagg gcgccgggcc | |
| 181 ggtgggagcc agcggcgcgc ggtgggaccc acggagcccc gcgacccgcc gagcctggag | |
| 241 ccgggccggc tcggggaagc cggctccagc ccggagcgaa cttcgcagcc cgtcgggggg | |
| 301 cggcggggag ggggcccgga gccggaggag ggggcggccg cgggcacccc cgcctgtgcc | |
| 361 ccggcgtccc cgggcaccat gctgtccaac tcccagggcc agagcccgcc ggtgccgttc | |
| 421 cccgccccgg ccccgccgcc gcagcccccc acccctgccc tgccgcaccc cccggcgcag | |
| 481 ccgccgccgc cgcccccgca gcagttcccg cagttccacg tcaagtccgg cctgcagatc | |
| 541 aagaagaacg ccatcatcga tgactacaag gtcaccagcc aggtcctggg gctgggcatc | |
| 601 aacggcaaag ttttgcagat cttcaacaag aggacccagg agaaattcgc cctcaaaatg | |
| 661 cttcaggact gccccaaggc ccgcagggag gtggagctgc actggcgggc ctcccagtgc | |
| 721 cccgacatcg tacggatcgt ggatgtgtac gagaatctgt acgcagggag gaagtgcctg | |
| 781 ctgattgtca tggaatgttt ggacggtgga gaactcttta gccgaatcca ggatcgagga | |
| 841 gaccaggcat tcacagaaag agaagcatcc gaaatcatga agagcatcgg tgaggccatc | |
| 901 cagtatctgc attcaatcaa cattgcccat cgggatgtca agcctgagaa tctcttatac | |
| 961 acctccaaaa ggcccaacgc catcctgaaa ctcactgact ttggctttgc caaggaaacc | |
| 1021 accagccaca actctttgac cactccttgt tatacaccgt actatgtggc tccagaagtg | |
| 1081 ctgggtccag agaagtatga caagtcctgt gacatgtggt ccctgggtgt catcatgtac | |
| 1141 attctgctgt gtgggtatcc ccccttctac tccaaccacg gccttgccat ctctccgggc | |
| 1201 atgaagactc gcatccgaat gggccagtat gaatttccca acccagaatg gtcagaagta | |
| 1261 tcagaggaag tgaagatgct cattcggaat ctgctgaaaa cagagcccac ccagagaatg | |
| 1321 accatcaccg agtttatgaa ccacccttgg atcatgcaat caacaaaggt ccctcaaacc | |
| 1381 ccactgcaca ccagccgggt cctgaaggag gacaaggagc ggtgggagga tgtcaaggag | |
| 1441 gagatgacca gtgccttggc cacaatgcgc gttgactacg agcagatcaa gataaaaaag | |
| 1501 attgaagatg catccaaccc tctgctgctg aagaggcgga agaaagctcg ggccctggag | |
| 1561 gctgcggctc tggcccactg agccaccgcg ccctcctgcc cacgggagga caagcaataa | |
| 1621 ctctctacag gaatatattt tttaaacgaa gagacagaac tgtccacatc tgcctcctct | |
| 1681 cctcctcagc tgcatggagc ctggaactgc atcagtgact gaattc | |

TABLE 4
Amino Acid Sequence of MK2 Isoform 2: Genbank Accession No. NP 116584

| | |
|---|---|
| 1 mlsnsqgqsp pvpfpapapp pqpptpalph ppaqpppppp qqfpqfhvks glqikknaii | (SEQ ID NO:4) |
| 61 ddykvtsqvl glgingkvlq ifnkrtqekf alkmlqdcpk arrevelhwr asqcpdivri | |
| 121 vdvyenlyag rkcllivmec ldggelfsri qdrgdqafte reaseimksi geaiqylhsi | |
| 181 niahrdvkpe nllytskrpn ailkltdfgf akettshnsl ttpcytpyyv apevlgpeky | |
| 241 dkscdmwslg vimyillcgy ppfysnhgla ispgmktrir mgqyefpnpe wsevseevkm | |
| 301 lirnllktep tqrmtitefm nhpwimqstk vpqtplhtsr vlkedkerwe dvkeemtsal | |
| 361 atmrvdyeqi kikkiedasn plllkrrkka raleaaalah | |

Transcript variant 1 includes an internal fragment in its 3′ region, which contains an upstream translational termination codon as compared to variant 2. Isoform 1 encoded by variant 1 is thus distinct from isoform 2 in C-terminus.

One of skill in the art will recognize that MK2 suitable for use in the present invention is desirably murine or human, but may include MK2 from any suitable organism. The protein and genomic sequences of these organisms are readily accessed via Genbank or The National Center for Biotechnology Information. As used in the present invention, “MK2 activity” includes, but is not limited to, MK2 enzymatic activity, such as kinase activity, and expression of MK2 in a cell.

Further, derivatives and homologues of MK2 may be used in the present invention. For example, nucleic acid sequences encoding MK2 of the present invention may be altered by substitutions, additions, or deletions that provide for functionally equivalent-conservative variants of MK2. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

Other conservative amino acid substitutions can be taken from the Table 5, below.

TABLE 5
Conservative amino acid replacements

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase protein stability; such analogs may contain, for example, one or more non-peptide bonds (which replace the peptide bonds) in the protein sequence. Also included are analogs that include residues other than naturally occurring L-amino acids, e.g., D-amino acids or non-naturally occurring or synthetic amino acids, e.g., β or γ amino acids.

MK2 as used in the present invention may be modified by, for example, phosphorylation, sulfation, acylation, or other protein modifications. It may also be modified with a label capable of providing a detectable signal, either directly or indirectly, including, but not limited to, radioisotopes and fluorescent compounds.

It will be apparent to one of skill in the art that conventional screening assays may be used in methods of the present invention for the identification of MK2 modulators.

By way of example only, assays suitable for use in the present invention include in-gel kinase assays and in vitro kinase assays, such as those set forth hereinbelow under “Materials and Methods”. These assays are useful for screening for potential MK2 modulatory compounds. Further, compounds found to affect MK2 activity may further be introduced into a suitable animal model, in order to study the activity of such compounds in vivo. Several downstream targets of MK2 have been reported to be phosphorylated and regulated by MK2, and are useful in such assays. These targets include heat shock protein 27 (Rouse et al., Cell 78:1027-1037 (1994)), lymphocyte-specific protein 1 (Huang et al., J. Biol. Chem. 272:17-19 (1997)), tyrosine hydroxylase (Thomas et al., Eur. J. Biochem. 247:1180-1189 (1997)) and 5-lipoxygenase (Werz et al., J. Biol. Chem. 277:14793-14800 (2002)).

In the present invention, techniques for screening large gene libraries may include cloning the gene library into replicable expression vectors, transforming appropriate cells with the resulting library of vectors, and expressing the genes under conditions for detection of a desired activity, e.g., binding of a ligand to MK2 in the present invention. Techniques known in the art are amenable to high throughput analysis for screening large numbers of sequences created, e.g., by random mutagenesis techniques. High throughput assays can be followed by secondary screens in order to identify further biological activities which will, e.g., allow one skilled in the art to differentiate agonists from antagonists. The type of a secondary screen used will depend on the desired activity that needs to be tested.

Drug screening assays are also provided in the present invention. By producing purified and recombinant MK2 of the present invention, or fragments thereof, one skilled in the art can use these to screen for drugs which are either agonists or antagonists of the normal cellular function or role in cellular signaling of MK2. In one aspect, the assay evaluates the ability of a compound to modulate binding between MK2 of the present invention and a naturally occurring ligand. The term “modulating” encompasses enhancement, diminishment, activation or inactivation of MK2 activity. Assays useful to identify ligands to MK2 of the present invention, including peptides, proteins, small molecules, and antibodies, that are capable of binding to MK2 and modulating its activity, are encompassed herein. A variety of assay formats may be used in the present invention and are known by those skilled in the art.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as primary screens in that they can be generated to permit rapid development and relatively easy detection of an alteration in a molecular target which is mediated by a test compound.

Compounds identified using assays, as discussed hereinabove, may be antagonists or agonists of MK2, and may bind to MK2, thereby modulating MK2 activity. Ligands to MK2 of the present invention, including peptides, proteins, small molecules, and antibodies, that are capable of binding to MK2 and modulating its activity, are encompassed herein. These compounds are useful in modulating the activity of MK2 and in treating MK2-associated disorders.

“MK2-associated disorders” refers to any disorder or disease state in which the MK2 protein plays a regulatory role in the metabolic pathway of that disorder or disease. As used herein the term “treating” refers to the alleviation of symptoms of a particular disorder in a patient, the improvement of an ascertainable measurement associated with a particular disorder, or the prevention of a particular immune, inflammatory or cellular response.

More specifically, compounds identified by assays of the present invention are useful to treat ischemia, including ischemia resulting from vascular occlusion, cerebral infarction, stroke, and related cerebral vascular diseases (including cerebrovascular accident and transient ischemic attack). Accordingly, the compounds may be used to treat myocardial infarction, coronary artery disease, non-Q wave MI, congestive heart failure, ventricular hypertrophy, cardiac arrhythmias, unstable angina, chronic stable angina, Prinzmetal's angina, high blood pressure, intermittent claudication, silent ischemia, and peripheral occlusive arterial disease (e.g., peripheral arterial disease, critical limb ischemia, such as critical leg ischemia, prevention of amputation, and prevention of cardiovascular morbidity such as MI, stroke or death).

Additionally, in view of their activity in treating ischemia, the compounds identified according to the present invention may be useful to treat symptoms or consequences occurring from thrombosis, atherosclerosis, peripheral arterial disease, and thrombotic or thromboembolic symptoms or consequences associated with and/or caused by one or more of the following: thromboembolic stroke (including that resulting from atrial fibrillation or from ventricular or aortic mural thrombus), venous thrombosis (including deep vein thrombosis), arterial thrombosis, cerebral thrombosis, pulmonary embolism, cerebral embolism, thrombophilia (e.g., Factor V Leiden, and homocystinenimia), coagulation syndromes and coagulopathies (e.g., disseminated intravascular coagulation), restenosis (e.g., following arterial injury induced endogenously or exogenously), atrial fibrillation and ventricular enlargement (including dilated cardiac myopathy and heart failure).

The compounds identified according to the present invention may be used to treat symptoms or consequences of atherosclerotic diseases and disorders, such as atherosclerotic vascular disease, atherosclerotic plaque rupture, atherosclerotic plaque formation, transplant atherosclerosis, and vascular remodeling atherosclerosis.

Compounds identified according to the present invention further may be used to treat symptoms or consequences of thrombotic or thromboembolic conditions associated with cancer, surgery, inflammation, systematic infection, artificial surfaces (such as stents, blood oxygenators, shunts, vascular access ports, vascular grafts, artificial valves, etc.), interventional cardiology such as percutaneous transluminal coronary angioplasty (PTCA), immobility, medication (such as oral contraceptives, hormone replacement therapy, and heparin), pregnancy and fetal loss, and diabetic complications including retinopathy, nephropathy, and neuropathy.

Compounds identified according to the present invention may also be used for the preservation of tissue, for example, the preservation of tissue as relates to organ transplantation and surgical manipulation. Such compounds may be used to treat diseases or disorders in other tissues or muscles that are associated with ischemic conditions and/or to enhance the strength or stability of tissue and muscles. For example, the compounds may be used to treat muscle cell damage and necrosis and/or to enhance athletes' performance.

Additional diseases and disorders that may be treated with compounds identified according to the present invention include CNS disorders associated with cerebral ischemia, such as cerebral infarction, cerebral edema and the like. Compounds identified according to the present invention may also be used for the treatment of neurotrauma and neurological diseases, such as Alzheimer's disease.

A compound which acts as a MK2 modulator may be administered for therapeutic use as a raw chemical or may be the active ingredient in a pharmaceutical formulation. Such formulations of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Compounds of the present invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents.

Such compounds may, for example, be administered in a form suitable for immediate release or extended release. Immediate release or extended release may be achieved by the use of suitable pharmaceutical compositions comprising compounds of the present invention, or, particularly in the case of extended release, by the use of devices such as subcutaneous implants or osmotic pumps. Compounds of the present invention may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art.

Compounds of the present invention may also be delivered through the oral cavity by sublingual and/or buccal administration. Molded tablets, compressed tablets or freeze-dried tablets are exemplary forms which may be used. Exemplary compositions include those formulating the compound(s) of the present invention with fast dissolving diluents such as mannitol, lactose, sucrose and/or cyclodextrins.

Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to MK2-associated disorders.

The compounds of the present invention may be employed alone or in combination with each other and/or other suitable therapeutic agents useful in the treatment of MK2-associated disorders, such as ischemia.

In another aspect, the present invention relates to the use of an isolated nucleic acid in “antisense” therapy. As used herein, “antisense” therapy refers to administration or in situ generation of oligonucleotides or their derivatives which specifically hybridize under cellular conditions with the cellular mRNA and/or genomic DNA encoding MK2 of the present invention so as to inhibit expression of the encoded protein, e.g., by inhibiting transcription and/or translation. In general, “antisense” therapy refers to the range of techniques generally employed in the art, and includes any therapy which relies on specific binding to oligonucleotide sequences.

Gene constructs useful in antisense therapy may be administered in any biologically effective carrier, e.g., any formulation or composition capable of effectively delivering a nucleic acid sequence to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the present invention.

In addition to viral transfer methods, non-viral methods may also be employed. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. Nucleic acid sequences may also be introduced to cell(s) by direct injection of the gene construct or by electroporation.

In clinical settings, the gene delivery systems can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

The following section sets forth materials and methods used in the present invention, and which were utilized in the Example set forth hereinbelow.

### Materials and Methods

1. Abbreviations

CBF=cerebral blood flow;

ERK=extracellular regulated kinase;

ECA=external common carotid;

ELISA=enzyme linked immunosorbent assay;

ICA=internal common carotid;

IL=interleukin;

LPS=lipopolysaccharide;

JNK/SAPK=c-jun N-terminal kinase/stress-activated protein kinase;

MAP=mitogen-activated protein;

MCA=middle cerebral artery;

MCAO=occlusion of the middle cerebral artery;

MK2=MAP kinase-activated protein kinase 2;

PBS=phosphate-buffered saline;

TNF=tumor necrosis factor;

TTC=2,3,5-triphenyltetrazolium chloride

2. Focal Brain Ischemia

MK2^{-/-} mice were on a mixed 129v×C57BL/6 background as described in Katlyarov et al. (Nat. Cell Biol. 1:94-97 (1999)) and the colony further expanded by Charles River Laboratories (Wilmington, Mass. 01887). Genotyping of MK2^{-/-} was carried out using a three primer PCR with the following oligonucleotides:

(SEQ ID NO:5)

5′-cgtgggggtggggtgacatgctggttgac (5′MK2)

(SEQ ID NO:6)

5′-ggtgtcaccttgacatcccggtgag (3′MK2)

(SEQ ID NO:7)

5′-tgctcgctcgatgcgatgtttcgc (Neo)

A fragment length of about 500 bp indicates wild type and 800 bp stands for the gene disruption.

Adult MK2^{-/-} and C57BL/6 mice (18-22 g, paired for gender and weight; Charles River Laboratories, Wilmington, Mass.) were used throughout the experiments. Animals were housed and cared for in accordance with the Guide for the Care and Use of Laboratory Animals [DHEW (DHHS) Publication No. (N1H) 85-23, revised 1996, Office of Science and Health Reports, DRR/NIH, Bethesda, Md. 20205]. Procedures using lab animals were approved by the Institutional Animal Care and Use Committee of Bristol-Myers Squibb Company.

Mice were anesthetized with gas inhalation comprised of a mixture of 30% oxygen (0.3 liter/min; Airgas East, Inc., Salem, N.H.) and 70% nitrous oxide (0.7 liter/min; Airgas East, Inc., Salem, N.H.). The gas was passed through an isoflurane vaporizer (VetEquip Inc., Pleasanton, Calif.) set to deliver 3-4% isoflurane (isoflurane (Hanna's Pharm Supply Co., Wilmington, Del.) during initial induction and 1.5-2% during surgery. Under such conditions, an incision of the skin was made directly on top of the right common carotid artery region, and the bifurcation of the external common carotid (ECA) and internal common carotid (ICA) was identified. A small incision was made on the ECA, and a 5-0 mono-filament suture (9-11 mm long with a round tip) (Sherwood Medical, St. Louis, Mo.) was thread into the ICA via the ECA. The suture was advanced towards the middle cerebral artery (MCA) to create focal ischemia. In the case of permanent brain ischemia, the suture was not removed, while the suture was removed 30 min after MCAO for transient brain ischemia. Sham-operation was performed using the same procedure except that no suture was inserted into the carotid artery. At the end of the study, mice were anesthetized with gas inhalation and forebrains were removed at various times following ischemia, reperfusion or sham surgery as indicated in each figure legend. For biochemical analysis, the entire ipsilateral and contralateral hemispheres were dissected and immediately frozen in liquid nitrogen and stored at −80° C. for later use.

To measure the infarct volume, brains were removed at 24 hr after MCAO and evaluated using 2,3,5-triphenyltetrazolium chloride (TTC) (Sigma Co., St. Louis, Mo.) staining of 2-mm thick brain slices. The stained brain tissue was fixed in 10% formalin in phosphate buffered saline (PBS) (VWR Scientific Products, West Chester, Pa.). The image was captured using a Microtek ScanMaker 4 DUO Scanner (MicroWarehouse Lakewood, N.J.) and quantitated using Image Pro Plus 4.1 software (Media Cybernetics, Silver Spring, Md.).

3. Neurological Deficits

Neurological deficits were examined at day 1 and 3 after MCAO (n=10) using a 5-point scale adapted and modified from Zhang et al. (Brain Res. 766:83-92 (1997)). Specifically, no neurological deficit=0; right Horner's syndrome counts 1 point; failure to extend left forelimb and hindlimb, 1 point each; turning to left, 1 point; and circling to left, 1 point.

4. Physiological Parameters

The physiological parameters were measured and confirmed under two anesthesia conditions, i.e., gas inhalation as described above and pentobarbital (50 mg/kg, i.p.) (Abbott Laboratories, North Chicago, Ill.). In randomly selected animals, regional cerebral blood flow (CBF) was measured with a Laser Doppler Perfusion Monitor (Moor Instruments Inc., Wilmington, Del.). After anesthesia, a small incision was made at the midpoint between the right orbit and the external auditory canal. The temporalis muscle was retracted and the underlying fascia cleared. The Laser Doppler probe was placed 1.5 mm posterior and 3.5 mm lateral to the Bregma on the ipsilateral hemisphere. CBF was carefully monitored (to avoid any large vessel) before, during (15 min) and after (30 min) MCAO. Relative CBF was calculated as percentage relative to levels prior to MCAO.

The arterial blood pressure and heart rate were measured by connecting a tubing through the femoral artery using an MP100 Workstation and analyzed using an AcqKnowledge software (BIOPAC Systems, Inc, Santa Barbara, Calif.) according to the manufacture's specification. Femoral arterial blood samples were analyzed for pH, oxygen (pO₂) and carbon dioxide (pCO₂) by direct collection through a PE-50 tubing into an i-STAT G3+ cartridge and processed with a portable clinical analyzer (Abbott Laboratories, Abbott Park, Ill.).

5. Real-time RT-PCR

Total RNA was isolated from ipsilateral and contralateral brain tissues (n=8) after transient MCAO or after sham-operation using an RNA isolation kit from Qiagen (Valencia, Calif.). The primers and probes (Table 6) used for real-time RT-PCR were designed using a Primer-Express 1.0 software from PE Applied Biosystems (Foster City, Calif.). The specificity of PCR primers for IL-1β, TNFα and a house keeping gene, rpL32, was tested using a standard PCR protocol in Perkin-Elmer thermocycler (Model 9600; Foster City, Calif.) prior to TaqMan quantitation and confirmed by gel electrophoresis.

PCR primers (F, forward; R, reverse) and probes were synthesized according to the mouse TNF-α (GenBank accession No. M13049), IL-1β (GenBank accession No. M15131) and rpL32 (GenBank accession No. AK002353) cDNA sequences, respectively. TaqMan probes contains 6-FAM for IL-1β and TNFα at 5′-end and VIC for the rpL32. All the probes have a quencher dye, 6-carboxy-tetramethyl-rhodamine (TAMRA), at the 3′ end.

TABLE 6
Primers and TaqMan probes used in the real-time PCR

| | | |
|---|---|---|
| Primer/Probe | Sequences | Position (bp) |

| | | |
|---|---|---|
| TNFα-F | 5′ tcatgcaccaccatcaagga (SEQ ID NO:8) | sense 1081-1100 |
| TNFα-R | 5′ gaggcaacctgaccactctcc (SEQ ID NO:9) | antisense 1181-1161 |
| TNFα-probe | 5′ aatgggctttccgaattcactggagc (SEQ ID NO:10) | sense 1105-1130 |
| IL-1β-F | 5′ acactccttagtcctcggcca (SEQ ID NO:11) | sense 976-996 |
| IL-1 β-R | 5′ ccatcagaggcaaggaggaa (SEQ ID NO:12) | antisense 1076-1057 |
| IL-1 β-probe | 5′ caggtcgctcagggtcacaagaaacc (SEQ ID NO:13) | sense 1000-1025 |
| rpL32-F | 5′ tgtcctctaagaaccgaaaagc (SEQ ID NO:14) | sense 360-381 |
| rpL32-R | 5′ cgttgggattggtgactctga (SEQ ID NO:15) | antisense 431-411 |
| rpL32-probe | 5′ ttgtagaaagagcagcacagctggcc (SEQ ID NO:16) | sense 384-409 |

Real-time PCR was performed as described in Wang et al. (J. Neurosci. Res. 59:238-246 (2000) with the following modifications: One-step RT-PCR was performed using a Gibco BRL PLATINUM Taq System (GIBCO BRL, Grand Island, N.Y.) according to the manufacturer's specification. The reaction started with 0.5-1 μg of total RNA at 25 μl reaction volume. The reaction mixture contained 12.5 μl of 2× Reaction Mix, 0.6 μl of 50 mM MgSO4, 0.125 μl RNase inhibitor, 0.5 μl each of the 10 μM forward and reverse primer, 0.5 μl of the 5 μM probe, and 0.3 μl of RT/Taq Mix. The mixture was incubated in 50° C. for 30 min, 95° C. for 5 min and then started the PCR cycles at 95° C. 15 seconds and 60° C. 60 seconds for 40 cycles. Each RT-PCR was done in duplicate and performed simultaneously. Data were analyzed using the Sequence Detector V1.6.3 program (Perkin-Elmer).

6. Enzyme Linked Immunosorbent Assay for IL-1β

Tissue lysate from ipsilateral and contralateral brain samples (15 hours after MCAO for the peak expression of IL-1β, n>7) were pulverized using a porcelain mortar and pestle under liquid nitrogen. The pulverized brain tissues were incubated in a lysis buffer (10 mM Tris pH.8.0, 150 mM NaCl, 1 mM EDTA, 1 mM PMSF, 1% Triton x-100) and 5 μl/ml of protease inhibitor cocktail (Sigma, P-8340) for one hour at 4 C°. After 10 min centrifugation at 10,000 g, the supernatant of tissue lysate was collected and aliquoted for enzyme linked immunosorbent assay (ELISA) and protein concentration measurement using a Bio-Rad DC Protein Assay kit (Bio-Rad, Hercules, Calif.). The levels of IL-1β protein in the brain tissue were measured using an ELISA kit for mouse IL-1β (Pierce Endogen, Rockford, Ill.) following the manufacturer's specification. Tissue extracts (50 μl) were applied to each well for the ELISA and the final measure was read out using a plate reader at 450 nm. The concentration of IL-1β protein in each sample was determined according to the standard (recombinant mouse IL-1β protein) provided with the kit. All the measured IL-1β, concentrations were at the linear part of the standard curve. Each sample was normalized by its total protein concentration in mg.

7. Western Blot Analysis

Western blot analysis was used to evaluate the levels of the active form of caspase-3 in MK2^{-/-} (n=8) and wild type (n=9) mice 24 hours after transient MCAO. The pulverized brain tissues were lysed and processed as described above in the Enzyme Linked Immunosorbent Assay for IL-1β Section. The soluble component of the tissue lysate was used for Western blot (100 μg protein/lane) using a mouse monoclonal IgG against caspase-3 (sc-7272) as described in Wang et al., Stroke 32:1020-1027 (2001) (Santa Cruz Biotechnology, Inc. Santa Cruz, Calif.). The blot was stripped and re-probed to a goat polyclonal anti-actin antibody (sc-1616) (Santa Cruz Biotechnology, Inc.).

8. Apoptosis Analysis

Apoptosis was measured by quantitating the DNA fragmentation in MK2^{-/-} (n=8) and wild type (n=9) mice 24 hours after transient MCAO using a Cell Death Detection ELISA kit (Roche Molecular Biochemicals, Indianapolis, Ind.). This sandwich-enzyme immunoassay provides a quantitative determination of histone-associated DNA fragments (mono- and oligo-nucleosomes) based on a photometric reaction using monoclonal antibodies directed against both DNA and histones. Frozen, pulverized brain tissue was lysed using the lysing buffer provided by the kit (30 minutes at room temperature) and pelleted (200×g). Aliquots of the supernatant were used in the assay according to the manufacturer's protocol.

9. Statistical Analysis

Data in text and figures are mean±standard errors for the indicated number (N) of animals. Statistical comparisons were made by analysis of variance (ANOVA; Fisher's protected least squares difference) and values were considered to be significant at p<0.05.

10. In-Gel Kinase Assay

Total cell lysates, corresponding to 0.5×106 cells, and MK2-IPs (corresponding to 2-5×106 cells) were loaded on 10% SDS/PAGE gels. A Mini Protean system (Bio-Rad) was used, and separation gels contained 0.15 mg/ml purified recombinant human 5-LO or HSP25. After electrophoresis, gels were washed 5×10 min at RT in 60-ml aliquots of buffer A [20% (vol/vol) isopropyl alcohol in 50 mM Tris·HCl (pH 8)] to remove SDS. Then, gels were washed 5×10 min at RT in 60-ml aliquots of buffer B (50 mM Tris·HCl, pH 8/1 mM DTT). Proteins in the gel were denatured by incubation for 1 h at RT in buffer C (50 mM Tris·HCl, pH 8/20 mM DTT/2 mM EDTA/6 M guanidine·HCl). To renature proteins, gels were washed once for 10 min at RT in 60 ml of buffer D (50 mM Tris·HCl, pH 8/1 mM DTT/2 mM EDTA/0.04% Tween 20) followed by overnight incubation in 300 ml of the same buffer at 4° C. with shaking. After preincubation at RT for 1 h in 30 ml of kinase buffer (20 mM Hepes, pH 7.6/20 mM MgC12/25 mM -glycerophosphate/10 mM 4-nitrophenylphosphate/2 mM DTT/0.2 mM Na3VO4), gels were finally incubated in 10 ml of kinase buffer containing 50 μM ATP and 10 μCi/ml [−32 P]ATP, for 1 h at 30° C. with shaking. To remove unreacted [−32 P]ATP, gels were washed in 50-ml aliquots of washing buffer [1% (wt/vol) sodium pyrophosphate and 5% (wt/vol) trichloroacetic acid] for 2 days with several buffer exchanges, followed by drying (in vacuo) and autoradiography.

11. In Vitro Kinase Assay

For in vitro phosphorylation, purified recombinant 5-LO or HSP25 (3 μg) was incubated with active MK2 or with MK2-IPs from cell lysates, in kinase buffer (25 mM Hepes, pH 7.5/25 mM MgC12/25 mM -glycerophosphate/2 mM DTT/0.1 mM Na3VO4) containing ATP (100 μM) and [−32 P]ATP (2 μCi/ml). The final volume was 20 μl, and incubation time was 30 min at 30° C. The reaction was terminated by addition of SDS-b and heating at 95° C. for 6 min. Samples were separated by SDS/PAGE (see “Western Blot”), and phosphorylated proteins were visualized by autoradiography of the dried gel.

### EXAMPLE

Using the materials and methods set forth above, ischemic brain injury was observed in MK2 deficient and wild type mice, following either transient or permanent MCAO. As indicated below, ischemic brain injury was significantly reduced in MK2 deficient mice compared to that of wild type mice.

Physiological parameters in MK2^{-/-} and wild type mice after cerebral ischemia were observed. Cerebral blood flow, heart rate, arterial blood pressure, pH, blood oxygen (pO2) and carbon dioxide (pCO2) were measured in MK2^{-/-} and wild type mice before and after transient MCAO (Table 7). Mice were subjected to 30 min MCAO followed by reperfusion. The physiological data were measured “before” (prior to MCAO), “during” (15 min after MCAO), or “after” (30 min of reperfusion) MCAO. CBF, cerebral blood flow (% arbitrary units; the relative arbitrary unit prior to MCAO was illustrated as 100%); HR, heart rate (per min); MABP, mean artery blood pressure (mmHg). *p<0.05, compared to the wild-type mice.

TABLE 7
Physiological conditions in the wild-type and MK2^{-/-} mice after
MCAO with reperfusion.

| | | | | MABP | pCO₂ | pO₂ | pH |
|---|---|---|---|---|---|---|---|
| Treatment | MCAO | CBF | HR | (min⁻¹) | (mmHg) | (mmHg) | (mmHg) |
| Wt: | | n = 7 | n = 6 | n = 6 | n = 6 | n = 6 | n = 6 |
| | before | 100 | 299 ± 41 | 84 ± 4 | 39 ± 4 | 114 ± 12 | 7.33 |
| | during | 23 ± 5 | 326 ± 18 | 82 ± 4 | 47 ± 3 | 93 ± 15 | 7.32 |
| | after | 37 ± 7 | 348 ± 45 | 87 ± 4 | 37 ± 1 | 121 ± 6 | 7.41 |
| MK2^{-/-}: | | n = 7 | n = 6 | n = 6 | n = 8 | n = 8 | n = 8 |
| | before | 100 | 376 ± 25 | 82 ± 8 | 42 ± 4 | 84 ± 13 | 7.34 |
| | during | 28 ± 6 | 373 ± 26 | 89 ± 4 | 56 ± 3 | 99 ± 9 | 7.27 |
| | after | 44 ± 5 | 434 ± 18 | 97 ± 3* | 44 ± 6 | 112 ± 9 | 7.34 |

No significant difference was observed in CBF, heart rate, and blood gases between MK2^{-/-} and wild type mice prior to and after MCAO. The only significant difference was the 11% increase in the mean arterial blood pressure in MK2^{-/-} mice compared to wild type mice 30 min after reperfusion (p<0.05; Table 7). However, this small increase in blood pressure is within the normal range in mice.

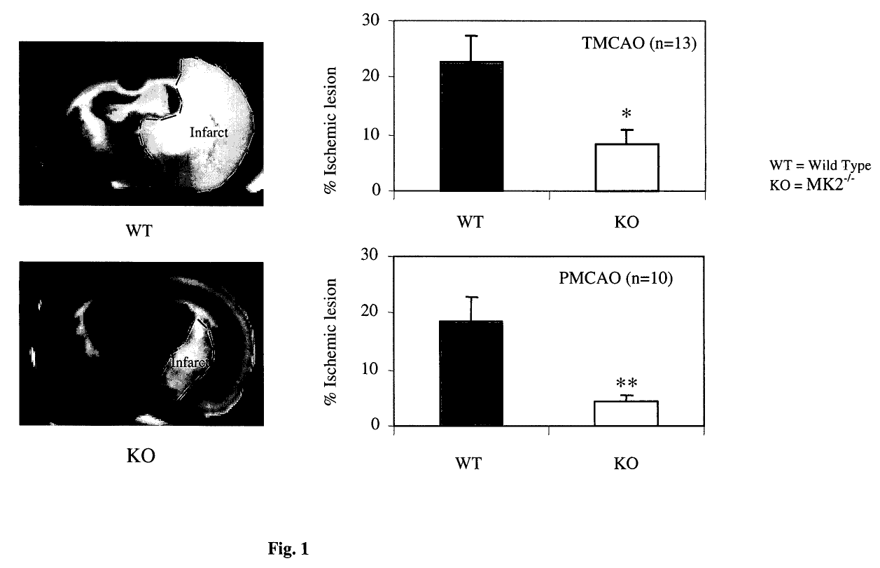

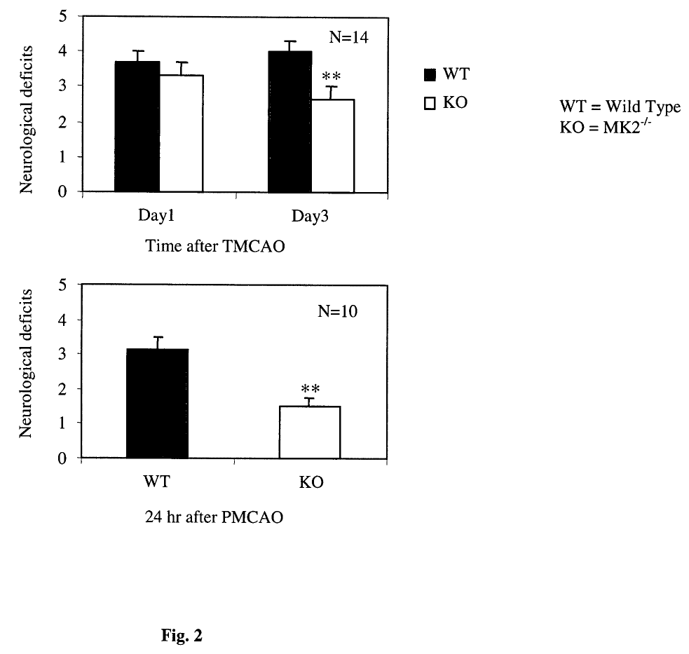

MK2^{-/-} was found to provide partial protection from ischemic brain injury. As shown in FIG. 1, significant reduction in infarct size was observed following transient (64% reduction, n=13, p<0.05) and permanent (76% reduction, n=10, p<0.01) MCAO in MK2^{-/-} mice compared with the paired wild type mice. The resistance of MK2^{-/-} mice to ischemic brain injury was also supported by the reduction in neurological deficits (FIG. 2). The neurological deficits were not significantly reduced in MK2^{-/-} mice until 3 days after transient MCAO (34% reduction compared to wild type mice, n=14, p<0.01). In contrast, a significant reduction in neurological deficits was observed 24 hours following permanent MCAO (52% reduction in MK2^{-/-} mice compared to the wild type mice, n=10, p<0.01) (FIG. 2). The three day neurological deficit data were not collected following permanent MCAO since these animals were processed for infarct size evaluation at 24 hours.

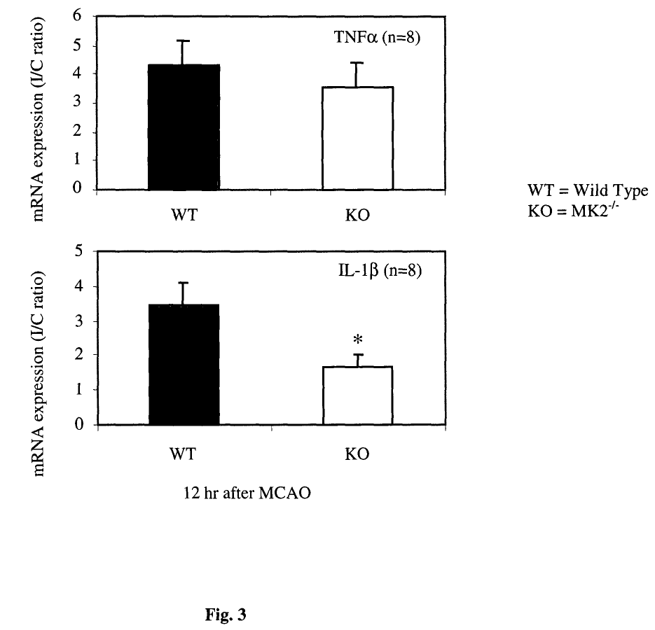

Cytokine gene expression in ischemic brain of MK2^{-/-} and wild type mice after MCAO was determined. FIG. 3 depicts the mRNA expression of two key inflammatory cytokines, IL-1β and TNFα, in MK2^{-/-} and wild type mice 12 hours after transient MCAO. Significant induction was observed for both cytokine mRNAs in the ipsilateral (ischemic) over the contralateral brain tissue in wild type mice (with 4.3- and 3.4-fold increase for TNFα and IL-1β mRNA, respectively). However, in MK2^{-/-} mice, significant induction was only seen in TNFα mRNA (3.6-fold increase in the ipsilateral brain tissue) but not IL-1 β mRNA (1.6-fold increase) after MCAO (FIG. 3). The levels of IL-1β mRNA expression in the ischemic brain tissue were significantly lower in MK2^{-/-} than in wild type mice (p<0.05, n=8).

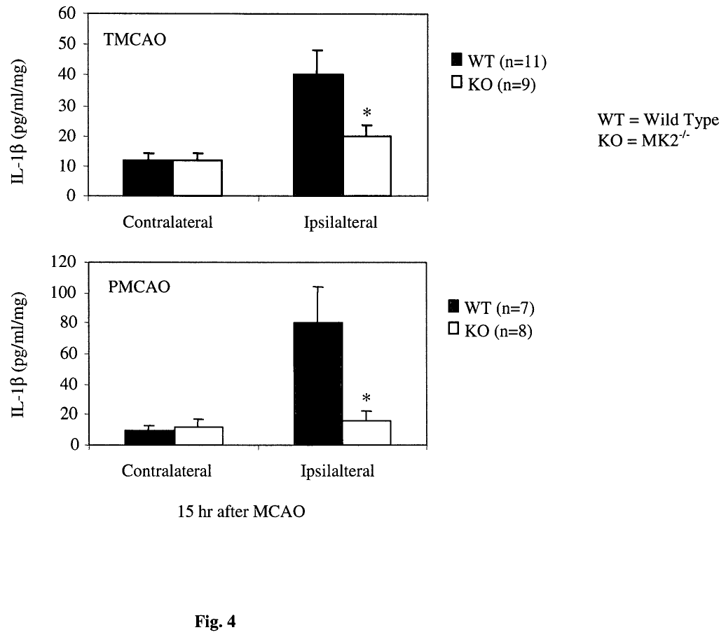

ELISA analysis showed that levels of IL-1β expression were increased for 3.3-fold (n=11, p<0.05) and 7.9-fold (n=7, p<0.01) in the ischemic brain tissue over the nonischemic (contralateral) tissue in wild type mice 15 hours after transient and permanent MCAO, respectively. Similar to its mRNA induction profile, the levels of IL-1β expression after brain ischemia were significantly lower in MK2^{-/-} mice (FIG. 4), i.e., only 49% (n=9, p<0.05) and 21% (n=8, p<0.05) compared to the wild type mice following transient and permanent MCAO, respectively.

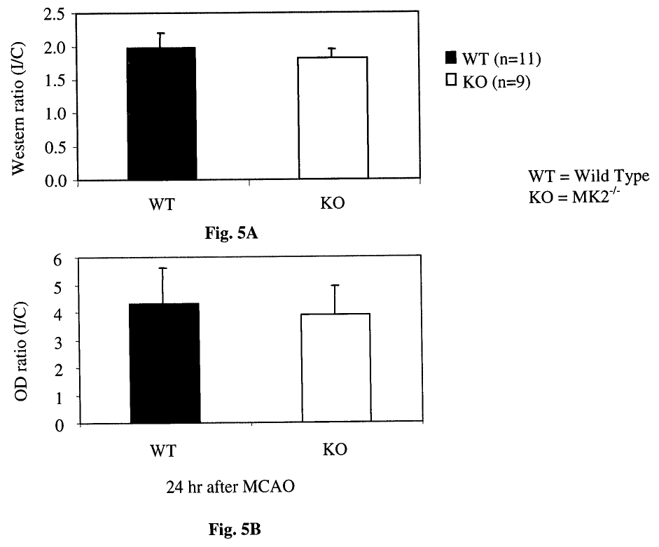

Comparative analysis of caspase-3 activation and apoptosis in MK2^{-/-} and wild type mice after MCAO was determined. Because MAP kinase has been implicated in cell survival as well as apoptosis following cerebral ischemic injury, evaluation of key markers of apoptosis, i.e., activation of caspase-3 (assessed for the expression of active caspase-3) and DNA fragmentation was conducted. Western analysis was used to detect the expression of active caspase-3 (p20) in the brain after MCAO. The levels of active caspase-3 in the ischemic brain was significantly elevated 24 hr after MCAO in both MK2^{-/-} and wild type mice, showing 1.8- and 2.0-fold increase, respectively, over the contralateral tissue. However, no significant difference was observed between these two experimental groups (FIG. 5A).

Similarly, while 3.9- and 4.3-fold increase in DNA fragmentation was observed in MK2^{-/-} and wild type mice, respectively, after cerebral ischemia, as evaluated by measurement of DNA fragmentation using an ELISA method, no significant difference was noted between these two groups (FIG. 5B).

### Discussion

As illustrated in the Example, above, ischemic brain injury was significantly reduced in MK2 deficient mice compared to that of wild type mice following either transient or permanent MCAO. Mice deficient in MK2 showed no difference in several key hemodynamic, hematologic and biochemical parameters as compared to wild type animals under normal conditions or post stroke, however they were protected from both transient and permanent MCAO, as evidenced by smaller infarct size and improved neurological function. The relative resistance of the MK2^{-/-} mice to stroke was manifested not only by reduction in infarct size but also by improvement in motor function (FIGS. 1 and 2).

Specifically, MK2^{-/-} mice subjected to focal ischemia markedly reduced infarct size by 64% and 76% following transient and permanent ischemia, respectively, compared to wild type mice. Furthermore, MK2^{-/-} mice had significant reduction in neurological deficits. Real-time polymerase chain reaction analysis identified a significant lower expression in interleukin-1β mRNA (53% reduction) but not in tumor necrosis factor-α mRNA in MK2^{-/-} mice over wild type animals after ischemic injury. The significant reduction in interleukin-1β was also confirmed in MK2^{-/-} mice by enzyme linked immunosorbent assay. The marked neuroprotection from ischemic brain injury in MK2^{-/-} mice was not associated with the alteration of hemodynamic or systemic variables, activation of caspase-3 and apoptosis. These results indicate the involvement of MAP kinase pathway in focal ischemic brain injury and indicate that this effect is associated with the expression of interleukin-1β in the ischemic brain tissue.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

What is claimed is:

1. A method of reducing ischemic injury in a mammal, comprising administering to said mammal a compound that reduces activity of MK2.

2. The method of claim 1, wherein said activity is MK2 expression.

3. The method of claim 1, wherein said ischemic injury is selected from the group consisting of cerebral ischemia, myocardial ischemia and critical limb ischemia.

4. The method of claim 1, wherein said compound inhibits transcription of MK2.

5. The method of claim 4, wherein said compound is an antisense nucleic acid.

6. The method of claim 5, wherein said antisense nucleic acid molecule comprises at least 10 nucleotides the sequence of which is complementary to an mRNA encoding an MK2 polypeptide.

7. The method of claim 5, wherein said antisense nucleic acid is a DNA, wherein transcription of said DNA yields nucleic acid product which is complementary to an mRNA encoding an MK2 polypeptide.

8. The method of claim 1, wherein said compound binds to a regulatory sequence operably linked to MK2.

9. A method of reducing ischemic injury in a mammal, comprising administering to said mammal an inhibitor of MK2 expression.

10. A method of reducing ischemic injury in a mammal, comprising administering to said mammal a compound that reduces activity of MK2.

11. A method for identifying a compound which inhibits MK2 expression in a cell, said method comprising the steps of:
(a) providing a cell that expresses MK2;
(b) culturing said cell in the presence of a test compound; and
(c) determining the level of expression of a MK2 in said cell,
wherein a decrease in said level of expression in the presence of said test compound compared to the level of expression in the absence of said test compound indicates that said test compound inhibits MK2 expression in said cell.

12. A compound identified by the method of claim 11.

13. The compound of claim 12, wherein said compound is a MK2 antagonist.

14. An assay for identifying a compound that modulates the activity of MK2, comprising:
(a) providing a cell expressing MK2;
(b) contacting said cell expressing MK2 with a test compound; and
(c) determining whether said test compound modulates the activity of MK2.

15. The assay of claim 14, wherein said assay is a cell-based assay.

16. The assay of claim 14, wherein said assay is a cell-free assay.

17. The assay of claim 16, wherein said cell-free assay is a ligand-binding assay.

18. The assay of claim 14, wherein said test compound modulates the activity of MK2.

19. The assay of claim 14, wherein said test compound is a MK2 antagonist.

20. The assay of claim 14, wherein said test compound is a MK2 agonist.

21. The assay of claim 14, wherein said test compound binds to MK2.

22. The assay of claim 14, wherein said assay is for identifying compounds which will be useful for the treatment of ischemic injury.

23. A compound identified by the assay of claim 14.

24. The compound of claim 23, wherein said compound is a MK2 antagonist.

25. A method for the treatment of ischemic injury, comprising administering to a patient in need thereof a therapeutically effective amount of a compound of claim 23.

26. A method for the treatment of ischemic injury, comprising:
(a) identifying a patient suffering from ischemic injury; and
(b) administering to said patient a therapeutically effective amount of a modulator of MK2.


SEQUENCE LISTING (16 sequences)

SEQ ID NO: 1 - DNA, 2263 bp, Human
gatatcacagcaacattgaaatgctaaaaagtttttaaacactctcaatttctaattcaccatgtcacagactggtgaaaaaaaaaaaaaaagcggccgcttccccccggccgggcccccgccgccccgcggtccccagagcgccaggcccccggggggagggagggagggcgccgggccggtgggagccagcggcgcgcggtgggacccacggagccccgcgacccgccgagcctggagccgggccggctcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtgagcagaggattctgtgttcctgtccaaactcagtgctgtttcttagaatccttttattccctgggtctctaatgggaccttaaagaccatctggtatcatcttctcattttgcagaagagaaactgaggcccagaggcggagggcagtctgctcaaggtcacgcagctggtgactggttggggcagaccggacccaggtttcctgactcctggcccaagtctcttcctcctatcctgcgggatcactggggggctctcagggaacagcagcagtgccatagccaggctctctgctgcccagcgctggggtgaggctgccgttgtcagcgtggaccactaaccagcccgtcttctctctctgctcccacccctgccgccctcaccctgcccttgttgtctctgtctctcacgtctctcttctgctgtctctcctacctgtcttctggctctctctgtacccttcctggtgctgccgtgcccccaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattc

SEQ ID NO: 2 - protein, 370 aa, Human
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKGCLHDKNSDQATWLTRL

SEQ ID NO: 3 - DNA, 1726 bp, Human
gatatcacagcaacattgaaatgctaaaaagtttttaaacactctcaatttctaattcaccatgtcacagactggtgaaaaaaaaaaaaaaagcggccgcttccccccggccgggcccccgccgccccgcggtccccagagcgccaggcccccggggggagggagggagggcgccgggccggtgggagccagcggcgcgcggtgggacccacggagccccgcgacccgccgagcctggagccgggccggctcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgccccgacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacattctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattc

SEQ ID NO: 4 - protein, 400 aa, Human
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPDIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 5 - DNA, 29 bp, Artificial
  Primer
cgtgggggtggggtgacatgctggttgac

SEQ ID NO: 6 - DNA, 25 bp, Artificial
  Primer
ggtgtcaccttgacatcccggtgag

SEQ ID NO: 7 - DNA, 24 bp, Artificial
  Primer
tgctcgctcgatgcgatgtttcgc

SEQ ID NO: 8 - DNA, 20 bp, Artificial
  Primer
tcatgcaccaccatcaagga

SEQ ID NO: 9 - DNA, 21 bp, Artificial
  Primer
gaggcaacctgaccactctcc

SEQ ID NO: 10 - DNA, 26 bp, Artificial
  Probe
aatgggctttccgaattcactggagc

SEQ ID NO: 11 - DNA, 21 bp, Artificial
  Primer
acactccttagtcctcggcca

SEQ ID NO: 12 - DNA, 20 bp, Artificial
  Primer
ccatcagaggcaaggaggaa

SEQ ID NO: 13 - DNA, 26 bp, Artificial
  Probe
caggtcgctcagggtcacaagaaacc

SEQ ID NO: 14 - DNA, 22 bp, Artificial
  Primer
tgtcctctaagaaccgaaaagc

SEQ ID NO: 15 - DNA, 21 bp, Artificial
  Primer
cgttgggattggtgactctga

SEQ ID NO: 16 - DNA, 26 bp, Artificial
  Probe
ttgtagaaagagcagcacagctggcc